(19) United States
(12) Patent Application Publication
(10) Pub. No.: US 2024/0335636 A1
(43) Pub. Date: Oct. 10, 2024

(54) Catheter-Disinfecting System and Methods Thereof

(71) Applicant: Bard Access Systems, Inc., Salt Lake City, UT (US)

(72) Inventors: Brendan Laine, Salt Lake City, UT (US); James J. Semler, Randolph, NJ (US)

(21) Appl. No.: 18/132,261

(22) Filed: Apr. 7, 2023

(51) Int. Cl. A61M 25/00

(52) U.S. Cl. CPC A61M 25/00; A61M 2025/0019

(57) ABSTRACT

A catheter-disinfecting system can include, in some embodiments, an introducer sheath and a germicidal irradiation means for irradiating a catheter tube of a catheter with germicidal radiation. The introducer sheath can include a sheath body configured to be percutaneously inserted into an insertion site of a patient. The germicidal irradiation means can be configured for irradiating the catheter tube as the catheter tube is inserted into a lumen of the introducer sheath, thereby disinfecting the catheter tube immediately before insertion into the insertion site. A method of disinfecting a catheter such as the foregoing catheter can include inserting the catheter tube into the introducer sheath while the introducer sheath, itself, is percutaneously inserted into the insertion site of the patient; and irradiating the catheter tube with the germicidal radiation while inserting the catheter tube into the introducer sheath.

### BACKGROUND

Currently, medical devices such as catheters are packaged and sterilized in bulk in their packaging using ethylene oxide or some other common, industrial-scale sterilization technique before they are distributed, sold, and used by clinicians. By way of a catheter for one of the foregoing medical devices, a clinician generally removes the catheter from its packaging and manipulates it in various ways according to procedure before inserting and placing the catheter in a patient. However, such manipulation compromises the sterility of the catheter, which can lead to contamination of the catheter with infection-causing bacteria or their endotoxemia-causing endotoxins. Therefore, it would be advantageous to disinfect the catheter and, by extension, any of a number of other similar medical devices, immediately before insertion into an insertion site or subsequent placement in the patient.

Disclosed herein are catheter-disinfecting systems and methods thereof for disinfecting medical devices such as catheters before insertion and placement in patients.

### SUMMARY

Disclosed herein is a catheter-disinfecting system including, in some embodiments, an introducer sheath and a germicidal irradiation means for irradiating a catheter tube of a catheter with germicidal radiation. The introducer sheath includes a sheath body and a sheath hub. The sheath body is configured to be percutaneously inserted into an insertion site of a patient. The sheath hub is coupled to a proximal portion of the sheath body. The germicidal irradiation means is configured for irradiating the catheter tube as the catheter tube is inserted into a lumen of the introducer sheath, thereby disinfecting the catheter tube immediately before insertion into the insertion site.

In some embodiments, the germicidal irradiation means includes one or more internal radiation sources incorporated into the introducer sheath. The one-or-more internal radiation sources are configured to both produce and emit the germicidal radiation into the lumen of the introducer sheath.

In some embodiments, the one-or-more internal radiation sources are incorporated into the sheath body for circumferentially irradiating the lumen of the introducer sheath.

In some embodiments, the one-or-more internal radiation sources are incorporated into a diffuser disposed in the sheath body for circumferentially irradiating the lumen of the introducer sheath from the diffuser of the sheath body.

In some embodiments, the one-or-more internal radiation sources are incorporated into the sheath body behind a diffuser for circumferentially irradiating the lumen of the introducer sheath through the diffuser of the sheath body.

In some embodiments, the diffuser includes a plurality of dimples configured to scatter the germicidal radiation transmitted into the diffuser out into the lumen of introducer sheath.

In some embodiments, the one-or-more internal radiation sources are incorporated into the sheath hub for circumferentially irradiating the lumen of the introducer sheath.

In some embodiments, the one-or-more internal radiation sources are incorporated into a diffuser disposed in the sheath hub for circumferentially irradiating the lumen of the introducer sheath from the diffuser of the sheath hub.

In some embodiments, the one-or-more internal radiation sources are incorporated into the sheath hub behind a diffuser for circumferentially irradiating the lumen of the introducer sheath through the diffuser of the sheath hub.

In some embodiments, the diffuser includes a plurality of dimples configured to scatter the germicidal radiation transmitted into the diffuser out into the lumen of introducer sheath.

In some embodiments, the catheter-disinfecting system further includes a sheath-hub attachment configured to removably couple to the sheath hub. The germicidal irradiation means includes one or more internal radiation sources incorporated into the sheath-hub attachment. The one-or-more internal radiation sources are configured to both produce and emit the germicidal radiation into a lumen of the sheath-hub attachment.

In some embodiments, the one-or-more internal radiation sources are incorporated into a sheath-hub-attachment body for circumferentially irradiating the lumen of the sheath-hub attachment. The lumen of the sheath-hub attachment is configured to lead into the lumen of the introducer sheath when the sheath-hub attachment is coupled to the sheath hub.

In some embodiments, the one-or-more internal radiation sources are incorporated into a diffuser disposed in the sheath-hub-attachment body for circumferentially irradiating the lumen of the sheath-hub attachment from the diffuser of the sheath-hub attachment.

In some embodiments, the one-or-more internal radiation sources are incorporated into the sheath-hub-attachment body behind a diffuser for circumferentially irradiating the lumen of the sheath-hub attachment through the diffuser of the sheath-hub attachment.

In some embodiments, the diffuser includes a plurality of dimples configured to scatter the germicidal radiation transmitted into the diffuser out into the lumen of the sheath-hub attachment.

In some embodiments, the catheter-disinfecting system further includes a sheath-hub attachment configured to removably couple to the sheath hub. The germicidal irradiation means includes one or more external radiation sources operably coupled by a waveguide to the sheath-hub attachment. The one-or-more external radiation sources are configured to produce the germicidal radiation. The waveguide is configured to transmit the germicidal radiation. And either the waveguide or a diffuser operably coupled to the waveguide is configured to emit the germicidal radiation into a lumen of the sheath-hub attachment.

In some embodiments, one or more waveguide termini are incorporated into a sheath-hub-attachment body for circumferentially irradiating the lumen of the sheath-hub attachment. The lumen of the sheath-hub attachment is configured to lead into the lumen of the introducer sheath when the sheath-hub attachment is coupled to the sheath hub.

In some embodiments, the one-or-more waveguide termini are incorporated into the diffuser disposed in the sheath-hub-attachment body for circumferentially irradiating the lumen of the sheath-hub attachment from the diffuser of the sheath-hub attachment.

In some embodiments, the one-or-more waveguide termini are incorporated into the sheath-hub-attachment body behind the diffuser for circumferentially irradiating the lumen of the sheath-hub attachment through the diffuser of the sheath-hub attachment.

In some embodiments, the diffuser includes a plurality of dimples configured to scatter the germicidal radiation transmitted into the diffuser out into the lumen of the sheath-hub attachment.

In some embodiments, the waveguide includes one or more optical fibers. The one-or-more waveguide termini are one or more termini of the one-or-more optical fibers, respectively.

In some embodiments, the catheter-disinfecting system further includes a sheath insert configured to removably insert into the introducer sheath. The germicidal irradiation means includes one or more external radiation sources operably coupled by a waveguide to the sheath insert. The one-or-more external radiation sources are configured to produce the germicidal radiation. The waveguide is configured to transmit the germicidal radiation. And either the waveguide or a diffuser is operably coupled to the waveguide configured to emit the germicidal radiation into a lumen of the sheath insert.

In some embodiments, one or more waveguide termini are incorporated into a sheath-insert body for circumferentially irradiating the lumen of the sheath insert. The lumen of the sheath insert is coaxial with the lumen of the introducer sheath when the sheath insert is inserted into the introducer sheath.

In some embodiments, the one-or-more waveguide termini are incorporated into the diffuser disposed in the sheath-insert body for circumferentially irradiating the lumen of the sheath insert from the diffuser of the sheath insert.

In some embodiments, the one-or-more waveguide termini are incorporated into the sheath-insert body behind the diffuser for circumferentially irradiating the lumen of the sheath insert through the diffuser of the sheath insert.

In some embodiments, the diffuser includes a plurality of dimples configured to scatter the germicidal radiation transmitted into the diffuser out into the lumen of the sheath insert.

In some embodiments, the waveguide includes one or more optical fibers. The one-or-more waveguide termini are one or more termini of the one-or-more optical fibers, respectively.

In some embodiments, the germicidal radiation is selected from broad spectrum ultraviolet (“UV”)-visible (“vis”) light, broad spectrum UV light, UVA light, UVB light, UVC light, blue light, and modulated light thereof. The modulated light is modulated with respect to frequency, power, duration, or a combination thereof.

In some embodiments, the introducer sheath is splittable for removing the introducer sheath from the insertion site without having to remove the catheter from the insertion site.

Also disclosed herein is a catheter-disinfecting system including, in some embodiments, a catheter, an introducer sheath, an excitation irradiation means for irradiating the catheter tube of the catheter with excitation radiation. The catheter includes a catheter tube. The catheter tube, an abluminal coating on the catheter tube, or both the catheter tube and the abluminal coating include a photosensitizer configured to produce disinfecting reactive oxygen species (“ROS”) when irradiated with the excitation radiation. The introducer sheath includes a sheath body and a sheath hub. The sheath body is configured to be percutaneously inserted into an insertion site of a patient. The sheath hub is coupled to a proximal portion of the sheath body. The excitation irradiation means is configured for irradiation the catheter tube as the catheter tube is inserted into a lumen of the introducer sheath, thereby producing the ROS and disinfecting the catheter tube immediately before insertion into the insertion site.

In some embodiments, the photosensitizer is incorporated into the catheter tube.

In some embodiments, the photosensitizer is incorporated into the abluminal coating on the catheter tube.

In some embodiments, the photosensitizer is selected from toluidine blue, methylene blue, rose bengal, and a porphyrin-based ROS-generating compound.

In some embodiments, the excitation irradiation means includes one or more internal radiation sources incorporated into the introducer sheath. The one-or-more internal radiation sources are configured to both produce and emit the excitation radiation into the lumen of the introducer sheath.

In some embodiments, the catheter-disinfecting system further includes a sheath-hub attachment configured to removably couple to the sheath hub. The excitation irradiation means includes one or more internal radiation sources incorporated into the sheath-hub attachment. The one-or-more internal radiation sources are configured to both produce and emit the excitation radiation into a lumen of the sheath-hub attachment.

In some embodiments, the catheter-disinfecting system further includes a sheath-hub attachment configured to removably couple to the sheath hub. The excitation irradiation means includes one or more external radiation sources operably coupled by a waveguide to the sheath-hub attachment. The one-or-more external radiation sources are configured to produce the excitation radiation. The waveguide is configured to transmit the excitation radiation. And either the waveguide or a diffuser operably coupled to the waveguide is configured to emit the excitation radiation into a lumen of the sheath-hub attachment.

In some embodiments, the catheter-disinfecting system further includes a sheath insert configured to removably insert into the introducer sheath. The excitation irradiation means includes one or more external radiation sources operably coupled by a waveguide to the sheath insert. The one-or-more external radiation sources are configured to produce the excitation radiation. The waveguide is configured to transmit the excitation radiation. And either the waveguide or a diffuser is operably coupled to the waveguide configured to emit the excitation radiation into a lumen of the sheath insert.

Also disclosed herein is a method of disinfecting a catheter. The method includes, in some embodiments, a catheter-inserting operation and a catheter-irradiating operation. The catheter-inserting operation includes inserting a catheter tube of the catheter into an introducer sheath, which introducer sheath is percutaneously inserted into an insertion site of a patient. The catheter-irradiating operation includes irradiating the catheter tube with radiation while inserting the catheter tube into a sheath body of the introducer sheath by way of a sheath hub. The irradiating of the catheter tube disinfects the catheter tube immediately before insertion into the insertion site.

In some embodiments, the irradiating operation includes irradiating the catheter tube with germicidal radiation. The germicidal radiation selected from broad spectrum UV-vis light, broad spectrum UV light, UVA light, UVB light, UVC light, blue light, and modulated light thereof. The modulated light is modulated with respect to frequency, power, duration, or a combination thereof.

In some embodiments, the irradiating operation includes irradiating the catheter tube with excitation radiation. The catheter tube or an abluminal coating on the catheter include a photosensitizer configured to produce disinfecting ROS when irradiated with the excitation radiation.

In some embodiments, the irradiating operation includes irradiating the catheter tube with one or more internal radiation sources incorporated into the introducer sheath. The one-or-more internal radiation sources are configured to both produce and emit the radiation into the lumen of the introducer sheath.

In some embodiments, the irradiating operation includes irradiating the catheter tube with one or more internal radiation sources incorporated into a sheath-hub attachment configured to removably couple to the sheath hub.

In some embodiments, the irradiating operation includes irradiating the catheter tube with one or more external radiation sources operably coupled by a waveguide to a sheath-hub attachment removably coupled to the sheath hub. The one-or-more external radiation sources are configured to produce the radiation. The waveguide is configured to transmit the radiation. And either the waveguide or a diffuser operably coupled to the waveguide configured to emit the radiation into a lumen of the sheath-hub attachment.

In some embodiments, the irradiating operation includes irradiating the catheter tube with one or more external radiation sources operably coupled by a waveguide to a sheath insert removably inserted into the introducer sheath. The one-or-more external radiation sources are configured to produce the radiation. The waveguide is configured to transmit the radiation. And either the waveguide or a diffuser operably coupled to the waveguide configured to emit the radiation into a lumen of the sheath insert.

These and other features of the concepts provided herein will become more apparent to those of skill in the art in view of the accompanying drawings and following description, which describe particular embodiments of such concepts in greater detail.

### DRAWINGS

FIG. 1 illustrates a catheter-disinfecting system in accordance with some embodiments.

FIG. 2 illustrates an introducer sheath and a sheath-hub attachment of the catheter-disinfecting system in accordance with some embodiments.

FIG. 3 illustrates the introducer sheath of the catheter-disinfecting system without the sheath-hub attachment in accordance with some embodiments.

FIG. 4 illustrates internal radiation sources incorporated into a sheath body of the introducer sheath, a sheath-hub-attachment body of the sheath-hub attachment, or a sheath-insert body of a sheath insert in accordance with some embodiments.

FIG. 5 illustrates a diffuser disposed in the introducer sheath in accordance with some embodiments.

FIG. 6 illustrates internal radiation sources incorporated into the sheath body of the introducer sheath or the sheath-hub-attachment body of the sheath-hub attachment behind a diffuser in accordance with some embodiments.

FIG. 7 illustrates internal radiation sources incorporated into the diffuser of the sheath body or sheath-hub-attachment body in accordance with some embodiments.

FIG. 8 illustrates internal radiation sources incorporated into the sheath-hub-attachment body of the sheath-hub attachment and the diffuser disposed in the sheath body of the introducer sheath in accordance with some embodiments.

FIG. 9 further illustrates the internal radiation sources incorporated into the sheath-hub-attachment body of the sheath-hub attachment and the diffuser disposed in the sheath body of the introducer sheath in accordance with some embodiments.

FIG. 10 illustrates the diffuser with a plurality of dimples configured to scatter radiation transmitted into the diffuser in accordance with some embodiments.

FIG. 11 illustrates a catheter including a catheter tube with a photosensitizer in accordance with some embodiments.

FIG. 12 illustrates production of disinfecting ROS upon irradiation of the catheter tube with the photosensitizer in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

Again, medical devices such as catheters are currently packaged and sterilized in bulk in their packaging using ethylene oxide or some other common, industrial-scale sterilization technique before they are distributed, sold, and used by clinicians. By way of a catheter for one of the foregoing medical devices, a clinician generally removes the catheter from its packaging and manipulates it in various ways according to procedure before inserting and placing the catheter in a patient. However, such manipulation compromises the sterility of the catheter, which can lead to contamination of the catheter with infection-causing bacteria or their endotoxemia-causing endotoxins. Therefore, it would be advantageous to disinfect the catheter and, by extension, any of a number of other similar medical devices, immediately before insertion into an insertion site or subsequent placement in the patient.

Disclosed herein are catheter-disinfecting systems and methods thereof for disinfecting medical devices such as catheters before insertion and placement in patients.

### Catheter-Disinfecting Systems

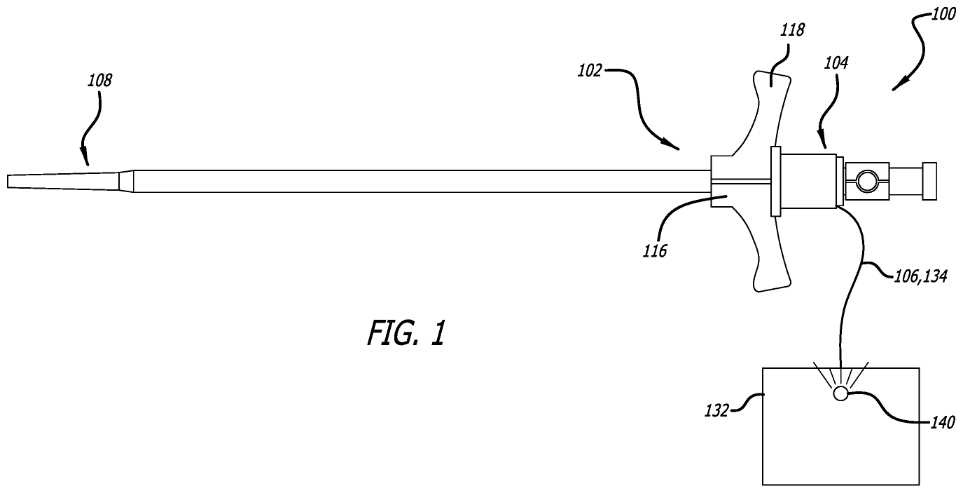

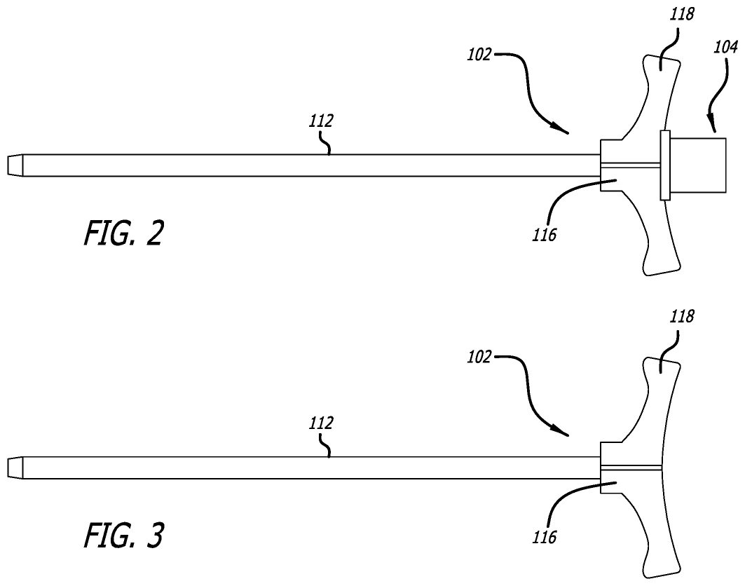

FIG. 1 illustrates a catheter-disinfecting system 100 in accordance with some embodiments. FIG. 2 illustrates an introducer sheath 102 and a sheath-hub attachment 104 of the catheter-disinfecting system 100 in accordance with some embodiments. And FIG. 3 illustrates the introducer sheath 102 of the catheter-disinfecting system 100 without the sheath-hub attachment 104 in accordance with some embodiments.

The catheter-disinfecting system 100 can include, depending upon embodiment, one or more components selected from the introducer sheath 102, a sheath insert 103, the sheath-hub attachment 104, and an irradiation means for irradiating a catheter tube 105 of a catheter 107 (e.g., a vascular catheter) with radiation. The irradiation means shown in FIG. 1 corresponds, in part, to the one-or-more external radiation sources 140 operably coupled by one or more waveguides 106 within suitable cabling to the sheath-hub attachment 104 set forth below, notably with an optional dilator 108 of the catheter-disinfecting system 100 removably inserted into a lumen 110 of the introducer sheath 102 by way of the sheath-hub attachment 104. However, as further set forth below, the irradiation means can vary in accordance with various embodiments.

The introducer sheath 102 can include a sheath body 112, optionally, a diffuser 114 disposed in or incorporated into the sheath body 112, for example, as a layer thereof, and a sheath hub 116 coupled to or around a proximal portion of the sheath body 112. The sheath body 112 is configured to be percutaneously inserted into an insertion site of a patient, optionally, over the dilator 108, which dilator 108 is configured to dilate a needle tract from the insertion site to a blood vessel to accommodate the sheath body 112. The sheath hub 116 can include a handle 118 having a pair of arms on opposite sides of the sheath hub 116 for manipulating the introducer sheath 102 while inserting the introducer sheath 102 into the insertion site or splitting the introducer sheath 102. Indeed, the introducer sheath 102 can be splittable for removing the introducer sheath 102 from the insertion site without having to remove the catheter 107 from the insertion site.

FIG. 5 illustrates, in an alternative to that set forth above and below, the sheath insert 103 removably inserted into the introducer sheath 102 in accordance with some embodiments. However, is should be understood that the sheath insert 103 is truncated such that a truncated distal portion of the sheath insert 103 is shown.

When present, the sheath insert 103 is configured to removably insert into the introducer sheath 102, for example, after removal of the dilator 108, such that a lumen 120 of the sheath insert 103 is coaxial with the lumen 110 of the introducer sheath 102 when the sheath insert 103 is inserted into the introducer sheath 102. The sheath insert 103 can form a part of the irradiation means, as set forth below. As such, the sheath insert 103 can include the diffuser 114 disposed in or incorporated into a sheath-insert body 122 of the sheath insert 103 in some embodiments.

When present, the sheath-hub attachment 104 is configured to removably couple to the sheath hub 116; however, the sheath-hub attachment 104 can be irremovable and splittable along with a remainder of the introducer sheath 102 in some embodiments. Again, the dilator 108 can be removably inserted into the lumen 110 of the introducer sheath 102 by way of the sheath-hub attachment 104. Indeed, a lumen 124 of the sheath-hub attachment 104 is configured to lead into the lumen 110 of the introducer sheath 102 when the sheath-hub attachment 104 is coupled to the sheath hub 116, thereby accommodating the dilator 108. The sheath-hub attachment 104 can form a part of the irradiation means, as set forth below. As such, the sheath-hub attachment 104 can include the diffuser 114 disposed in or incorporated into a sheath-hub-attachment body 126 of the sheath-hub attachment 104 in some embodiments.

The irradiation means can include a germicidal irradiation means configured for irradiating the catheter tube 105 of the catheter 107 with germicidal radiation as the catheter tube 105 is inserted into the lumen 110 of the introducer sheath 102, thereby disinfecting the catheter tube 105 immediately before insertion into the insertion site. Such germicidal radiation can include broad spectrum UV-vis light, broad spectrum UV light, UVA light, UVB light, UVC light, broad spectrum visible light, violet light, blue light, green light, yellow light, orange light, red light, or modulated light thereof, wherein the modulated light is modulated with respect to wavelength or frequency, power, including ramping the power, duration, including pulse durations when pulsing the modulated light, or a combination thereof. And such germicidal radiation can be provided by the one-or-more internal radiation sources 128 or the one-or-more external radiation sources 140 as set forth below with sufficient wavelength or frequency, power, and duration to effect disinfection.

The irradiation means can alternatively include an excitation irradiation means configured for irradiating the catheter tube 105 of the catheter 107 with excitation radiation as the catheter tube 105 is inserted into the lumen 110 of the introducer sheath 102, thereby disinfecting the catheter tube 105 immediately before insertion into the insertion site. Notably, the excitation radiation is paired with the catheter 142 set forth below having the catheter tube 144 or the abluminal coating thereon with the photosensitizer. The photosensitizer is configured to produce disinfecting ROS for disinfecting the catheter tube 144 when the catheter tube 144 or the abluminal coating thereon is irradiated with the excitation radiation. Such excitation radiation can include broad spectrum UV-vis light, broad spectrum UV light, UVA light, UVB light, UVC light, broad spectrum visible light, violet light, blue light, green light, yellow light, orange light, red light or modulated light thereof, wherein the modulated light is modulated with respect to wavelength or frequency, power, including ramping the power, duration, including pulse durations when pulsing the modulated light, or a combination thereof. And such excitation radiation can be provided by the one-or-more internal radiation sources 128 or the one-or-more external radiation sources 140 as set forth below with sufficient wavelength or frequency, power, and duration to effect disinfection.

The one-or-more internal radiation sources 128 or the one-or-more external radiation sources 140 as set forth below can be one or more semiconductor light sources independently selected from at least light-emitting diodes (“LEDs”), lasers, or superluminescent diodes (“SLDs”), which SLDs combine high-power and brightness characteristics of lasers with the low-power characteristic of LEDs.

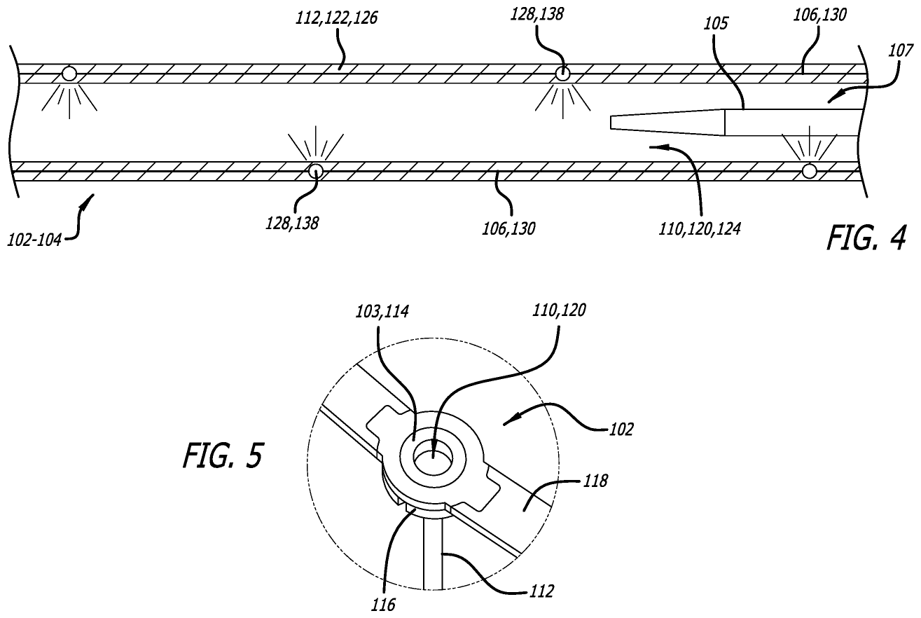

FIG. 4 illustrates internal radiation sources 128 incorporated into the sheath body 112 of the introducer sheath 102, the sheath-hub-attachment body 126 of the sheath-hub attachment 104, or the sheath-insert body 122 of the sheath insert 103 in accordance with some embodiments.

As shown, the irradiation means can include one or more internal radiation sources 128 incorporated into the introducer sheath 102, wherein the one-or-more internal radiation sources 128 are configured to both produce and emit the radiation into the lumen 110 of the introducer sheath 102. In particular, the one-or-more internal radiation sources 128 can be incorporated into the sheath body 112 for circumferentially irradiating the lumen 110 of the introducer sheath 102. Because the sheath hub 116 is coupled to or around the proximal portion of the sheath body 112, the one-or-more internal radiation sources 128 can be at least partially incorporated into the sheath hub 116 as well for circumferentially irradiating the lumen 110 of the introducer sheath 102.

As further shown, the irradiation means can include the one-or-more internal radiation sources 128 incorporated into the sheath-hub attachment 104, wherein the one-or-more internal radiation sources 128 are configured to both produce and emit the radiation into the lumen 124 of the sheath-hub attachment 104. In particular, the one-or-more internal radiation sources 128 can be incorporated into the sheath-hub-attachment body 126 for circumferentially irradiating the lumen 124 of the sheath-hub attachment 104.

As further shown, the irradiation means can include the one-or-more internal radiation sources 128 incorporated into the sheath insert 103, wherein the one-or-more internal radiation sources 128 are configured to both produce and emit the radiation into the lumen 120 of the sheath insert 103. In particular, the one-or-more internal radiation sources 128 can be incorporated into the sheath-insert body 122 for circumferentially irradiating the lumen 120 of the sheath insert 103. Notably, FIG. 7 illustrates, in an alternative to that set forth above and below, the internal radiation sources 128 incorporated into the sheath-insert body 122 of the sheath insert 103 and the sheath insert 103 inserted into the introducer sheath 102.

Notably, the one-or-more internal radiation sources 128 incorporated into the sheath body 112 of the introducer sheath 102, the sheath-hub-attachment body 126 of the sheath-hub attachment 104, or the sheath-insert body 122 of the sheath insert 103 can be controlled and powered over one or more wires 130, respectively, the one-or-more wires 130 operably connected to a controller 132 for control and powering the one-or-more internal radiation sources 128 over an external cable 134. (See, e.g., FIG. 1 with the understanding the external radiation source is absent in the foregoing embodiment.)

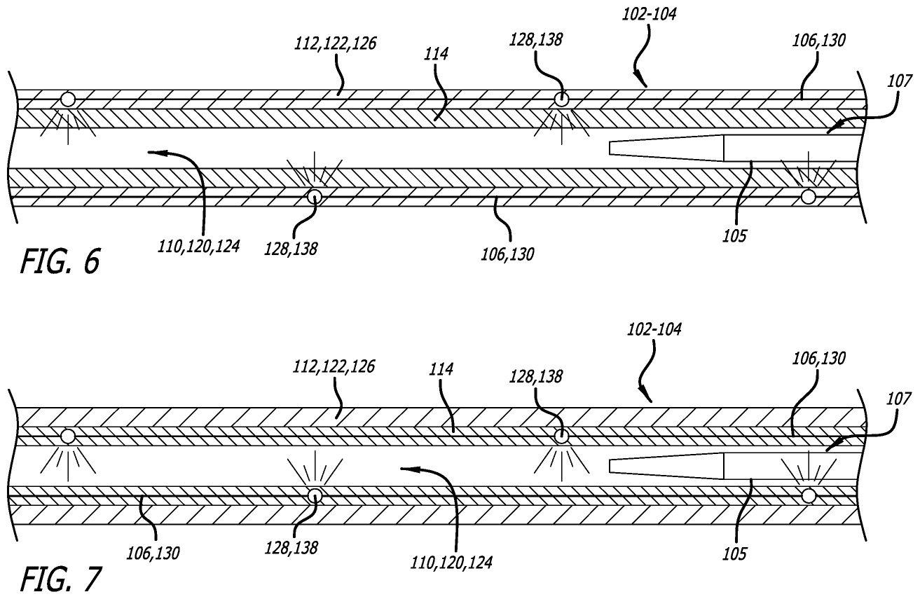

FIG. 5 illustrates the diffuser 114 disposed in the introducer sheath 102 in accordance with some embodiments. Notably, the diffuser 114 can be likewise disposed in the sheath-hub attachment 104 in accordance with some embodiments. FIG. 6 illustrates the internal radiation sources 128 incorporated into the sheath body 112 of the introducer sheath 102, the sheath-hub-attachment body 126 of the sheath-hub attachment 104, or the sheath-insert body 122 of the sheath insert 103 behind the diffuser 114 in accordance with some embodiments.

As shown, the irradiation means can include the one-or-more internal radiation sources 128 incorporated into the sheath body 112 behind the diffuser 114, wherein the one-or-more internal radiation sources 128 are configured to produce the radiation, the diffuser 114 is configured to transmit the radiation therethrough, and the diffuser 114 is configured to emit the radiation into the lumen 110 of the introducer sheath 102 for circumferentially irradiating the lumen 110 of the introducer sheath 102 through the diffuser 114 of the sheath body 112. Because the sheath hub 116 is coupled to or around the proximal portion of the sheath body 112, the one-or-more internal radiation sources 128 can be at least partially incorporated into the sheath hub 116 as well for circumferentially irradiating the lumen 110 of the introducer sheath 102 through the diffuser 114 of the sheath hub 116.

As further shown, the irradiation means can include the one-or-more internal radiation sources 128 incorporated into the sheath-hub-attachment body 126 behind the diffuser 114, wherein the one-or-more internal radiation sources 128 are configured to produce the radiation, the diffuser 114 is configured to transmit the radiation therethrough, and the diffuser 114 is configured to emit the radiation into the lumen 124 of the sheath-hub attachment 104 for circumferentially irradiating the lumen 124 of the sheath-hub attachment 104 through the diffuser 114 of the sheath-hub attachment 104.

As further shown, the irradiation means can include the one-or-more internal radiation sources 128 incorporated into the sheath-insert body 122 behind the diffuser 114, wherein the one-or-more internal radiation sources 128 are configured to produce the radiation, the diffuser 114 is configured to transmit the radiation therethrough, and the diffuser 114 is configured to emit the radiation into the lumen 120 of the sheath insert 103 for circumferentially irradiating the lumen 120 of the sheath insert 103 through the diffuser 114 of the sheath insert 103.

Notably, the one-or-more internal radiation sources 128 incorporated into the sheath body 112 of the introducer sheath 102 or the sheath-hub-attachment body 126 of the sheath-hub attachment 104 can be controlled and powered over the one-or-more wires 130, respectively, the one-or-more wires 130 operably connected to the controller 132 for control and powering the one-or-more internal radiation sources 128 over the external cable 134. (See, e.g., FIG. 1 with the understanding the external radiation source is absent in the foregoing embodiment.)

FIG. 7 illustrates the internal radiation sources 128 incorporated into the diffuser 114 of the sheath body 112, the sheath-hub-attachment body 126, or the sheath-insert body 122 in accordance with some embodiments.

As shown, the irradiation means can include the one-or-more internal radiation sources 128 incorporated into the diffuser 114 disposed in the sheath body 112, wherein the one-or-more internal radiation sources 128 are configured to produce the radiation, the diffuser 114 is configured to transmit the radiation therethrough, and the diffuser 114 is configured to emit the radiation into the lumen 110 of the introducer sheath 102 for circumferentially irradiating the lumen 110 of the introducer sheath 102 from the diffuser 114 of the sheath body 112. Because the sheath hub 116 is coupled to or around the proximal portion of the sheath body 112, the one-or-more internal radiation sources 128 can be at least partially located within the sheath hub 116 as well for circumferentially irradiating the lumen 110 of the introducer sheath 102 from the diffuser 114 of the sheath hub 116.

As further shown, the irradiation means can include the one-or-more internal radiation sources 128 incorporated into the diffuser 114 disposed in the sheath-hub-attachment body 126, wherein the one-or-more internal radiation sources 128 are configured to produce the radiation, the diffuser 114 is configured to transmit the radiation therethrough, and the diffuser 114 is configured to emit the radiation into the lumen 124 of the sheath-hub attachment 104 for circumferentially irradiating the lumen 124 of the sheath-hub attachment 104 from the diffuser 114 of the sheath-hub attachment 104.

As further shown, the irradiation means can include the one-or-more internal radiation sources 128 incorporated into the diffuser 114 disposed in the sheath-insert body 122, wherein the one-or-more internal radiation sources 128 are configured to produce the radiation, the diffuser 114 is configured to transmit the radiation therethrough, and the diffuser 114 is configured to emit the radiation into the lumen 120 of the sheath insert 103 for circumferentially irradiating the lumen 120 of the sheath insert 103 from the diffuser 114 of the sheath insert 103.

Notably, the one-or-more internal radiation sources 128 incorporated into the diffuser 114 can be controlled and powered over the one-or-more wires 130, respectively, the one-or-more wires 130 operably connected to the controller 132 for control and powering the one-or-more internal radiation sources 128 over the external cable 134. (See, e.g., FIG. 1 with the understanding the external radiation source is absent in the foregoing embodiment.)

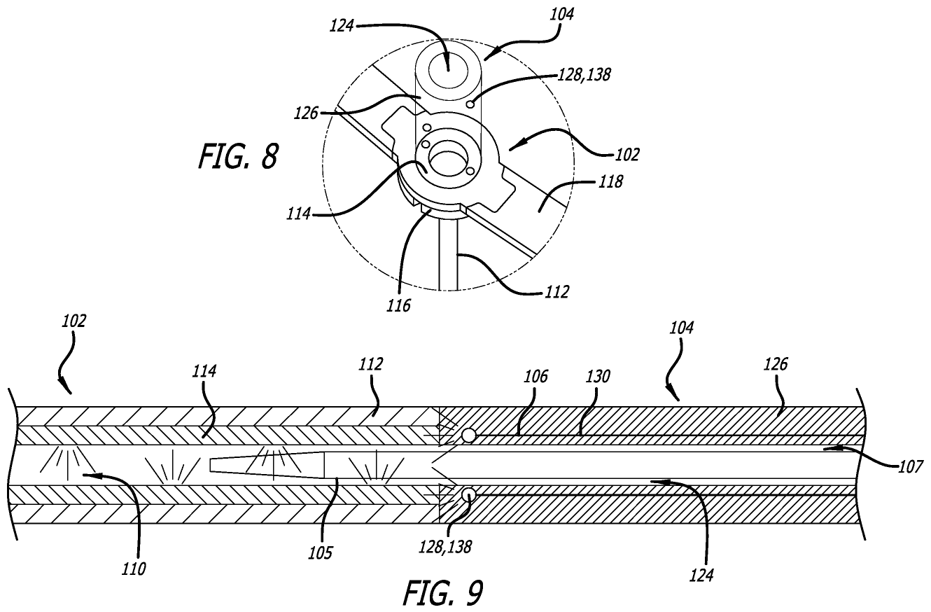

FIGS. 8 and 9 illustrate the internal radiation sources 128 incorporated into the sheath-hub-attachment body 126 of the sheath-hub attachment 104 and the diffuser 114 disposed in the sheath body 112 of the introducer sheath 102 in accordance with some embodiments.

As further shown, the irradiation means can include the one-or-more internal radiation sources 128 incorporated into the sheath-hub-attachment body 126 of the sheath-hub attachment 104, wherein the one-or-more internal radiation sources 128 are configured to both produce and emit the radiation into the diffuser 114 disposed in the sheath body 112 of the introducer sheath 102. Like that set forth above, the diffuser 114 is configured to transmit the radiation therethrough, and the diffuser 114 is configured to emit the radiation into the lumen 110 of the introducer sheath 102 for circumferentially irradiating the lumen 110 of the introducer sheath 102 through the diffuser 114 of the sheath body 112.

Notably, embodiments of the catheter-disinfecting system 100 in which the internal or external radiation sources 128 or 140 are at least external to the introducer sheath 102 can be advantageous in that the component can be reused as capital equipment. With respect to the foregoing sheath-hub attachment 104, for example, it includes the one or more internal radiation sources 128 which are external to the introducer sheath 102. This can be advantageous in that the sheath-hub attachment 104, itself, can be disinfected and reused as capital equipment while the introducer sheath 102 is disposed as single-use, disposable equipment.

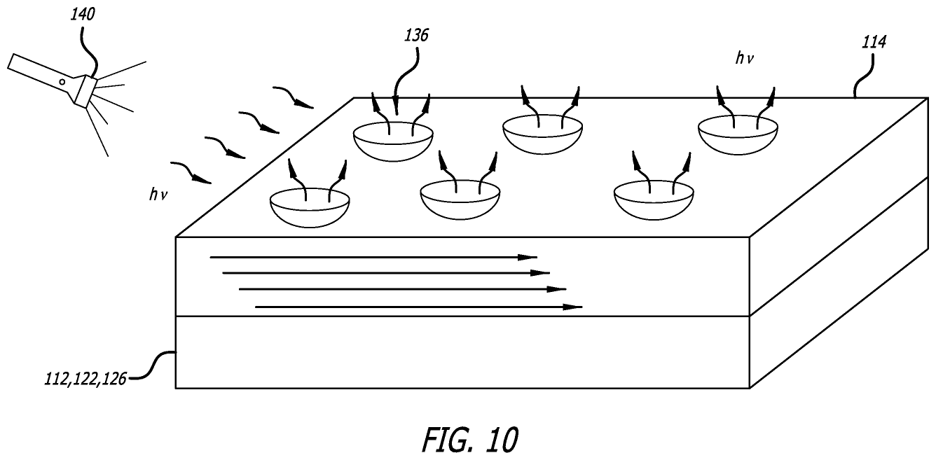

FIG. 10 illustrates the diffuser 114 with a plurality of dimples 136 configured to scatter radiation transmitted into the diffuser 114 in accordance with some embodiments.

As shown, the diffuser 114 disposed in the introducer sheath 102, the sheath-hub attachment 104, or the sheath insert 103 or otherwise incorporated, integrated, or the like into the introducer sheath 102, the sheath-hub attachment 104, or the sheath insert 103 as a layer thereof can include the plurality of dimples 136. The plurality of dimples 136 can open toward or face the lumen 110, 120, or 124 of the introducer sheath 102, the sheath-hub attachment 104, or the sheath insert 103, the plurality of dimples 136 thereby configured to scatter the radiation transmitted into the diffuser 114 out into the lumen 110, 120, or 124 of introducer sheath 102, the sheath-hub attachment 104, or the sheath insert 103.

FIG. 4 illustrates, in an alternative to that set forth above, waveguide termini 138 incorporated into the sheath body 112 of the introducer sheath 102, the sheath-hub-attachment body 126 of the sheath-hub attachment 104, or the sheath-insert body 122 of the sheath insert 103 in accordance with some embodiments including one or more external radiation sources 140 such as that shown in FIG. 1.

As shown, the irradiation means can include one or more waveguide termini 138 incorporated into the introducer sheath 102, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 110 of the introducer sheath 102. In particular, the one-or-more waveguide termini 138 can be incorporated into the sheath body 112 for circumferentially irradiating the lumen 110 of the introducer sheath 102. Because the sheath hub 116 is coupled to or around the proximal portion of the sheath body 112, the one-or-more waveguides 106 or the one-or-more waveguide termini 138 thereof can be at least partially incorporated into the sheath hub 116 as well for circumferentially irradiating the lumen 110 of the introducer sheath 102.

As further shown, the irradiation means can include the one-or-more waveguide termini 138 incorporated into the sheath-hub attachment 104, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 124 of the sheath-hub attachment 104. In particular, the one-or-more waveguide termini 138 can be incorporated into the sheath-hub-attachment body 126 for circumferentially irradiating the lumen 124 of the sheath-hub attachment 104.

As further shown, the irradiation means can include the one-or-more waveguide termini 138 incorporated into the sheath insert 103, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 120 of the sheath insert 103. In particular, the one-or-more waveguide termini 138 can be incorporated into the sheath-insert body 122 for circumferentially irradiating the lumen 120 of the sheath insert 103. Notably, FIG. 7 illustrates, in an alternative to that set forth above, the waveguide termini 138 incorporated into the sheath-insert body 122 of the sheath insert 103 and the sheath insert 103 inserted into the introducer sheath 102.

Notably, the one-or-more waveguides 106 can include one or more optical fibers, optionally, with cladding over each optical fiber of the one-or-more optical fibers and a polymeric coating over the cladding of each optical fiber of the one-or-more optical fibers, the one-or-more optical fibers packaged in suitable cabling, particularly with respect to any external waveguide like that shown in FIG. 1. Indeed, any internal waveguide such as that shown in FIGS. 4, 6, and 7 can be disposed in the sheath body 112 of the introducer sheath 102, the sheath-hub-attachment body 126 of the sheath-hub attachment 104, or the sheath-insert body 122 of the sheath insert 103 such as by extrusion or molding thereover. Further, the one-or-more waveguide termini 138 can respectively be one or more optical-fiber termini of the one-or-more optical fibers, wherein each terminus of the one-or-more optical-fiber termini is optionally disposed in an optical-fiber ferrule.

Again, FIG. 5 illustrates the diffuser 114 disposed in the introducer sheath 102 in accordance with some embodiments. Notably, the diffuser 114 can be likewise disposed in the sheath-hub attachment 104 in accordance with some embodiments. FIG. 6 illustrates in an alternative to that set forth above, the waveguide termini 138 incorporated into the sheath body 112 of the introducer sheath 102, the sheath-hub-attachment body 126 of the sheath-hub attachment 104, or the sheath-insert body 122 of the sheath insert 103 behind the diffuser 114 in accordance with some embodiments including the one-or-more external radiation sources 140 such as that shown in FIG. 1.

As shown, the irradiation means can include the one-or-more waveguide termini 138 incorporated into the sheath body 112 behind the diffuser 114, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 110 of the introducer sheath 102 for circumferentially irradiating the lumen 110 of the introducer sheath 102 through the diffuser 114 of the sheath body 112. Because the sheath hub 116 is coupled to or around the proximal portion of the sheath body 112, the one-or-more waveguides 106 or the one-or-more waveguide termini 138 thereof can be at least partially incorporated into the sheath hub 116 as well for circumferentially irradiating the lumen 110 of the introducer sheath 102 through the diffuser 114 of the sheath hub 116.

As further shown, the irradiation means can include the one-or-more waveguide termini 138 incorporated into the sheath-hub-attachment body 126 behind the diffuser 114, wherein the one-or-more external radiation sources 140 are configured to produce the radiation, one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 124 of the sheath-hub attachment 104 for circumferentially irradiating the lumen 124 of the sheath-hub attachment 104 through the diffuser 114 of the sheath-hub attachment 104.

As further shown, the irradiation means can include the one-or-more waveguide termini 138 incorporated into the sheath-insert body 122 behind the diffuser 114, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 120 of the sheath insert 103 for circumferentially irradiating the lumen 120 of the sheath insert 103 through the diffuser 114 of the sheath insert 103.

Notably, the one-or-more waveguides 106 can include one or more optical fibers, optionally, with cladding over each optical fiber of the one-or-more optical fibers and a polymeric coating over the cladding of each optical fiber of the one-or-more optical fibers, the one-or-more optical fibers packaged in suitable cabling, particularly with respect to any external waveguide like that shown in FIG. 1. Indeed, any internal waveguide such as that shown in FIGS. 4, 6, and 7 can be disposed in the sheath body 112 of the introducer sheath 102, the sheath-hub-attachment body 126 of the sheath-hub attachment 104, or the sheath-insert body 122 of the sheath insert 103 behind the diffuser 114 such as by extrusion or molding thereover. Further, the one-or-more waveguide termini 138 can respectively be one or more optical-fiber termini of the one-or-more optical fibers, wherein each terminus of the one-or-more optical-fiber termini is optionally disposed in an optical-fiber ferrule.

FIG. 7 illustrates, in an alternative to that set forth above, the waveguide termini 138 incorporated into the diffuser 114 of the sheath body 112, the sheath-hub-attachment body 126, or the sheath-insert body 122 in accordance with some embodiments.

As shown, the irradiation means can include the one-or-more waveguide termini 138 incorporated into the diffuser 114 disposed in the sheath body 112, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 110 of the introducer sheath 102 for circumferentially irradiating the lumen 110 of the introducer sheath 102 from the diffuser 114 of the sheath body 112. Because the sheath hub 116 is coupled to or around the proximal portion of the sheath body 112, the one-or-more waveguide termini 138 can be at least partially located within the sheath hub 116 as well for circumferentially irradiating the lumen 110 of the introducer sheath 102 from the diffuser 114 of the sheath hub 116.

As further shown, the irradiation means can include the one-or-more waveguide termini 138 incorporated into the diffuser 114 disposed in the sheath-hub-attachment body 126, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 124 of the sheath-hub attachment 104 for circumferentially irradiating the lumen 124 of the sheath-hub attachment 104 from the diffuser 114 of the sheath-hub attachment 104.

As further shown, the irradiation means can include the one-or-more waveguide termini 138 incorporated into the diffuser 114 disposed in the sheath-insert body 122, wherein the one-or-more external radiation sources 140 are configured to produce the radiation, the one-or-more waveguides 106 are operably coupled to the one-or-more external radiation sources 140 and configured to transmit the radiation, and the one-or-more waveguides 106 or the one-or-more waveguide termini 138 thereof are configured to emit the radiation into the lumen 120 of the sheath insert 103 for circumferentially irradiating the lumen 120 of the sheath insert 103 from the diffuser 114 of the sheath insert 103.

Notably, the one-or-more waveguides 106 can include one or more optical fibers, optionally, with cladding over each optical fiber of the one-or-more optical fibers and a polymeric coating over the cladding of each optical fiber of the one-or-more optical fibers, the one-or-more optical fibers packaged in suitable cabling, particularly with respect to any external waveguide like that shown in FIG. 1. Indeed, any internal waveguide such as that shown in FIGS. 4, 6, and 7 can be disposed in the diffuser 114 such as by extrusion or molding thereover. Further, the one-or-more waveguide termini 138 can respectively be one or more optical-fiber termini of the one-or-more optical fibers, wherein each terminus of the one-or-more optical-fiber termini is optionally disposed in an optical-fiber ferrule.

Lastly, any component of the catheter-disinfecting system 100 selected from the introducer sheath 102, the sheath insert 103, and the sheath-hub attachment 104 can include a material, a coating, a covering, or the like configured to absorb any outgoing radiation as a protecting means for protecting clinicians from exposure to the radiation. As such, whether the radiation is the germicidal radiation or the excitation radiation, the catheter-disinfecting system 100 can be configured to limit it to disinfecting the catheter tube 105 of the catheter 107 immediately before insertion into the insertion site.

### Catheters

As set forth above, the irradiation means can include the germicidal irradiation means or the excitation irradiation means. While any catheter or catheter tube thereof can be irradiated with the germicidal irradiation to disinfect the catheter tube immediately before insertion into the insertion site, the catheter 142 set forth below or a catheter likewise including the photosensitizer thereof is used with the excitation irradiation means to disinfect the catheter tube 144 immediately before insertion into the insertion site. Being that the catheter 142 is paired with the excitation irradiation, the catheter 142, too, can be part of the catheter-disinfecting system 100 in some embodiments.

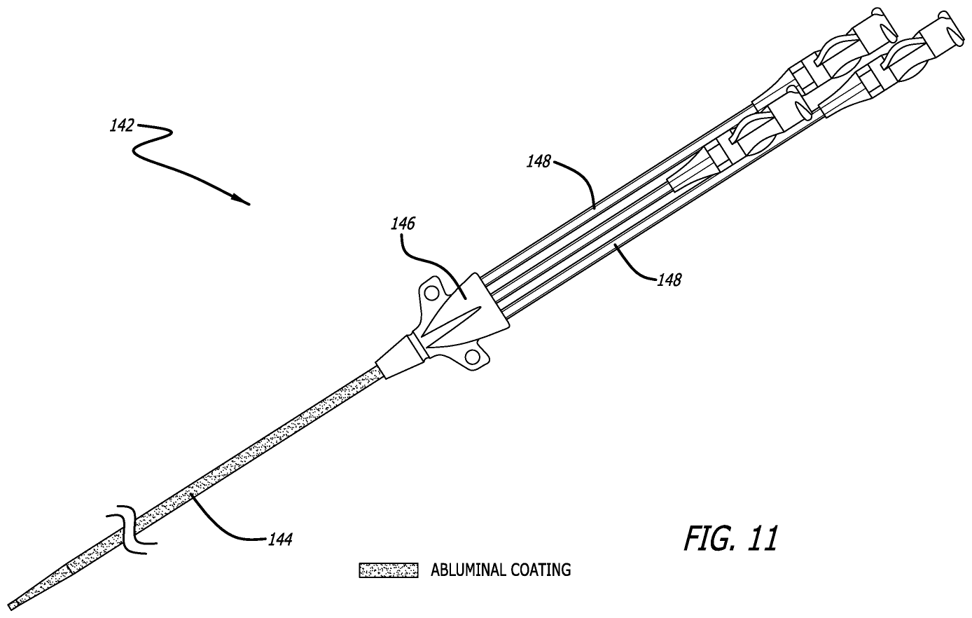

FIG. 11 illustrates a catheter 142 including a catheter tube 144 with a photosensitizer in accordance with some embodiments.

As shown, the catheter 142 can include the catheter tube 144, a catheter hub 146, and one or more extension legs 148 operably connected in the foregoing order. Indeed, the catheter tube 144 can include a proximal end portion disposed in the catheter hub 146, and each extension leg of the one-or-more extension legs 148 can include a distal end portion disposed in the catheter hub 146.

The catheter tube 144, an abluminal coating on the catheter tube 144, or both the catheter tube 144 and the abluminal coating can include a photosensitizer configured to produce disinfecting ROS when irradiated with the excitation radiation. Indeed, the photosensitizer can be incorporated into the catheter tube 144 during extrusion by admixing the photosensitizer with beads of a resin during the extrusion, or the photosensitizer can be incorporated into the catheter tube 144 during coextrusion by admixing the photosensitizer with beads of a first resin and coextruding the beads of the first resin over beads of a second resin during the coextrusion. Additionally or alternatively, the photosensitizer can be incorporated into the abluminal coating during coating by mixing the photosensitizer into a coating composition and applying the coating composition over an abluminal surface of the catheter tube 144. Such a photosensitizer can include, but is not limited to, toluidine blue, methylene blue, rose bengal, a porphyrin-based ROS-generating compound, or a combination thereof. Even porphyrin-based compounds within bacterial cells can be used to generate ROS, for example, with the blue light, which allows the excitation radiation to be used with the catheter 107 in some embodiments.

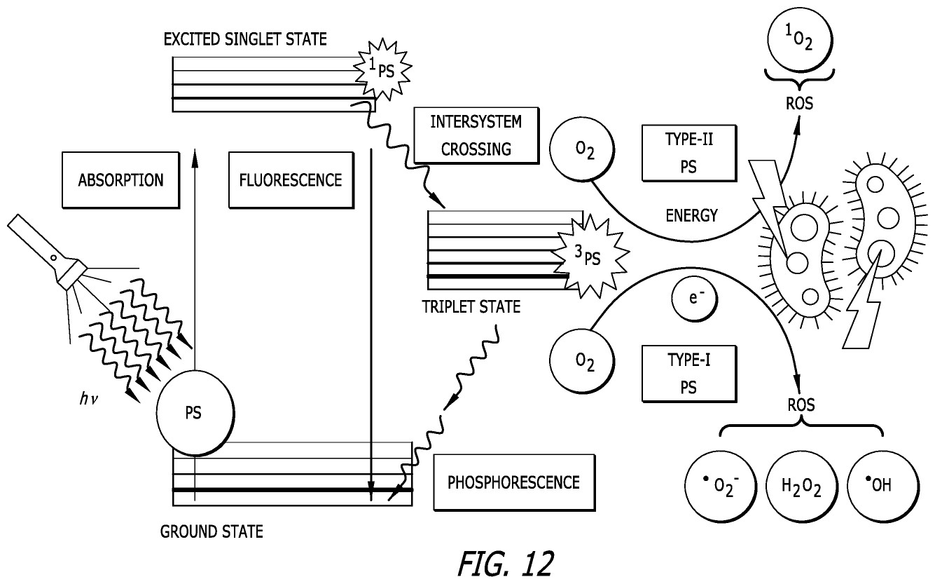

FIG. 12 illustrates production of disinfecting ROS upon irradiation of the catheter tube 144 with the photosensitizer in accordance with some embodiments.

As shown, upon the excitation irradiation means irradiating the catheter tube 144 as the catheter tube 144 is inserted into the lumen 110 of the introducer sheath 102, the photosensitizer, or some population of the photosensitizer, enters an excited singlet state. While the photosensitizer can lose energy by way of fluorescence, some population of the photosensitizer in the excited single state alternatively enters an excited triplet state through intersystem crossing. And, depending upon whether the photosensitizer is a type-I photosensitizer or a type-II photosensitizer, the photosensitizer in its excited triplet state reacts with oxygen by way of electron transfer or energy transfer to produce the disinfecting ROS. Indeed, the type-I photosensitizer in its excited triplet state reacts with oxygen by way of electron transfer to produce superoxide (^{•}O₂⁻) by reduction of oxygen, hydrogen peroxide (H₂O₂) by disproportionation of the superoxide, and hydroxyl radical (^{•}OH) by reduction of the hydrogen peroxide. The type-II photosensitizer in its excited triplet state reacts with oxygen by way of energy transfer to produce singlet oxygen (¹O₂). The disinfecting ROS, in turn, lead to bacterial death and inactivation of bacterial endotoxins by various reaction mechanisms involving the ROS.

### Methods

Methods include is a method of disinfecting a catheter (e.g., the catheter 107 or 142). Such a method includes, in some embodiments, a catheter-inserting operation and a catheter-irradiating operation.

The catheter-inserting operation can include inserting the catheter tube 105 or 144 of the catheter 107 or 142 into the introducer sheath 102 when introducer sheath 102 is percutaneously inserted into an insertion site of a patient.

The catheter-irradiating operation can include irradiating the catheter tube 105 or 144 with radiation while inserting the catheter tube 105 or 144 into the sheath body 112 of the introducer sheath 102 by way of the sheath hub 116. As set forth above, the radiation can be the germicidal radiation or the excitation radiation, though, in some embodiments, both the germicidal radiation and the excitation radiation can be used in the catheter-irradiating operation to disinfect the catheter tube 144 in accordance with two modalities. When both the germicidal radiation and the excitation radiation are used, wavelength ranges of the germicidal radiation and the excitation radiation can be separate and distinct from each other, partially overlap, or completely overlap, in which latter case the germicidal radiation and the excitation radiation are the same. It follows that when the wavelength ranges of the germicidal radiation and the excitation radiation are separate and distinct from each other or partially overlap, the internal or external radiation sources 128 or 140 are present as a plurality thereof, each radiation source independently configured to produce the germicidal radiation or the excitation radiation, or the internal or external radiation sources 128 or 140 are present as a single internal or external radiation source 128 or 140 configured to modulate between the germicidal radiation and the excitation radiation. That said, combinations of the foregoing are also possible. Regardless, the irradiating of the catheter tube 105 or 144 with the radiation disinfects the catheter tube 105 or 144 immediately before insertion into the insertion site. Not only does catheter-irradiating operation eliminate any time for additional bacteria or their endotoxins to accumulate on the catheter tube 105 or 144, but at no point after the catheter tube 105 or 144 is disinfected is it able to become contaminated until the catheter tube 105 or 144 is extracted from the patient.

### EXAMPLES

Table 1 set forth below provides some microbial reduction data from testing of various substrates under conditions ranging from dark, ambient light, and irradiation with UV light. Sample “E UV” represents a regular polyurethane coupon as an example portion of a catheter tube irradiated with UV for 15 minutes. As shown, there was between a 93 and 100% reduction in colony forming units (“CFUs”) depending upon the microorganism (i.e., Staphylococcus aureus, Pseudomonas aeruginosa, and Candida albicans). It should be understood that increases in light intensity enable sterilization at higher levels within a shorter, more clinically relevant, time periods.

TABLE 1
Microbial reduction data.

| | | | Summary | | | | | |
|---|---|---|---|---|---|---|---|---|
| | | | Staphylococcus | | Pseudomonas | | | |
| | | | aureus | | aeruginosa | | Candida albicans | |
| | | | 15-Minute treatment | | | | | |
| | | | Biofilm | | Biofilm | | Biofilm | |
| | | | Comparison | | | | | |
| | | | LogR | % Kill | LogR | % Kill | LogR | % Kill |
| E Dark | Vs. | C Amb | 0.31 | 51.43 | −1.22 | −1569.99 | −0.25 | −77.46 |
| | | C UV | 4.66 | 100.00 | 1.17 | 93.27 | 5.74 | 100.00 |
| | | C Dark | 0.00 | −0.60 | −2.16 | −14267.99 | 0.07 | 15.85 |
| | | D Amb | 0.08 | 16.52 | −0.80 | −524.80 | −0.21 | −60.38 |
| | | D UV | 2.28 | 99.48 | 1.17 | 93.27 | 5.74 | 100.00 |
| | | D Dark | 0.45 | 65.54 | −0.49 | −206.41 | 0.17 | 32.87 |
| | | E Amb | −0.11 | −29.70 | 1.17 | 93.27 | 0.05 | 10.29 |
| | | E UV | 3.44 | 99.96 | 1.17 | 93.27 | 4.94 | 100.00 |

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A catheter-disinfecting system, comprising:
an introducer sheath including:
a sheath body configured to be percutaneously inserted into an insertion site of a patient; and
a sheath hub coupled to a proximal portion of the sheath body; and
a germicidal irradiation means for irradiating a catheter tube of a catheter with germicidal radiation as the catheter tube is inserted into a lumen of the introducer sheath, thereby disinfecting the catheter tube immediately before insertion into the insertion site.

2. The catheter-disinfecting system of claim 1, wherein the germicidal irradiation means includes one or more internal radiation sources incorporated into the introducer sheath, the one-or-more internal radiation sources configured to both produce and emit the germicidal radiation into the lumen of the introducer sheath.

3. The catheter-disinfecting system of claim 2, wherein the one-or-more internal radiation sources are incorporated into the sheath body for circumferentially irradiating the lumen of the introducer sheath.

4. The catheter-disinfecting system of claim 3, wherein the one-or-more internal radiation sources are incorporated into a diffuser disposed in the sheath body for circumferentially irradiating the lumen of the introducer sheath from the diffuser of the sheath body.

5. The catheter-disinfecting system of claim 3, wherein the one-or-more internal radiation sources are incorporated into the sheath body behind a diffuser for circumferentially irradiating the lumen of the introducer sheath through the diffuser of the sheath body.

6. The catheter-disinfecting system of claim 4, wherein the diffuser includes a plurality of dimples configured to scatter the germicidal radiation transmitted into the diffuser out into the lumen of introducer sheath.

7. The catheter-disinfecting system of claim 2, wherein the one-or-more internal radiation sources are incorporated into the sheath hub for circumferentially irradiating the lumen of the introducer sheath.

8. The catheter-disinfecting system of claim 7, wherein the one-or-more internal radiation sources are incorporated into a diffuser disposed in the sheath hub for circumferentially irradiating the lumen of the introducer sheath from the diffuser of the sheath hub.

9. The catheter-disinfecting system of claim 7, wherein the one-or-more internal radiation sources are incorporated into the sheath hub behind a diffuser for circumferentially irradiating the lumen of the introducer sheath through the diffuser of the sheath hub.

10. The catheter-disinfecting system of claim 8, wherein the diffuser includes a plurality of dimples configured to scatter the germicidal radiation transmitted into the diffuser out into the lumen of introducer sheath.

11. The catheter-disinfecting system of claim 1, further comprising a sheath-hub attachment configured to removably couple to the sheath hub, the germicidal irradiation means including one or more internal radiation sources incorporated into the sheath-hub attachment, and the one-or-more internal radiation sources configured to both produce and emit the germicidal radiation into a lumen of the sheath-hub attachment.

12. The catheter-disinfecting system of claim 11, wherein the one-or-more internal radiation sources are incorporated into a sheath-hub-attachment body for circumferentially irradiating the lumen of the sheath-hub attachment, the lumen of the sheath-hub attachment configured to lead into the lumen of the introducer sheath when the sheath-hub attachment is coupled to the sheath hub.

13. The catheter-disinfecting system of claim 12, wherein the one-or-more internal radiation sources are incorporated into a diffuser disposed in the sheath-hub-attachment body for circumferentially irradiating the lumen of the sheath-hub attachment from the diffuser of the sheath-hub attachment.

14. The catheter-disinfecting system of claim 12, wherein the one-or-more internal radiation sources are incorporated into the sheath-hub-attachment body behind a diffuser for circumferentially irradiating the lumen of the sheath-hub attachment through the diffuser of the sheath-hub attachment.

15. The catheter-disinfecting system of claim 13, wherein the diffuser includes a plurality of dimples configured to scatter the germicidal radiation transmitted into the diffuser out into the lumen of the sheath-hub attachment.

16. The catheter-disinfecting system of claim 1, further comprising a sheath-hub attachment configured to removably couple to the sheath hub, the germicidal irradiation means including one or more external radiation sources operably coupled by a waveguide to the sheath-hub attachment, the one-or-more external radiation sources configured to produce the germicidal radiation, the waveguide configured to transmit the germicidal radiation, and either the waveguide or a diffuser operably coupled to the waveguide configured to emit the germicidal radiation into a lumen of the sheath-hub attachment.

17. The catheter-disinfecting system of claim 16, wherein one or more waveguide termini are incorporated into a sheath-hub-attachment body for circumferentially irradiating the lumen of the sheath-hub attachment, the lumen of the sheath-hub attachment configured to lead into the lumen of the introducer sheath when the sheath-hub attachment is coupled to the sheath hub.

18. The catheter-disinfecting system of claim 17, wherein the one-or-more waveguide termini are incorporated into the diffuser disposed in the sheath-hub-attachment body for circumferentially irradiating the lumen of the sheath-hub attachment from the diffuser of the sheath-hub attachment.

19. The catheter-disinfecting system of claim 17, wherein the one-or-more waveguide termini are incorporated into the sheath-hub-attachment body behind the diffuser for circumferentially irradiating the lumen of the sheath-hub attachment through the diffuser of the sheath-hub attachment.

20. The catheter-disinfecting system of claim 18, wherein the diffuser includes a plurality of dimples configured to scatter the germicidal radiation transmitted into the diffuser out into the lumen of the sheath-hub attachment.

21. The catheter-disinfecting system of claim 17, wherein the waveguide includes one or more optical fibers, the one-or-more waveguide termini being one or more termini of the one-or-more optical fibers, respectively.

22. The catheter-disinfecting system of claim 1, further comprising a sheath insert configured to removably insert into the introducer sheath, the germicidal irradiation means including one or more external radiation sources operably coupled by a waveguide to the sheath insert, the one-or-more external radiation sources configured to produce the germicidal radiation, the waveguide configured to transmit the germicidal radiation, and either the waveguide or a diffuser operably coupled to the waveguide configured to emit the germicidal radiation into a lumen of the sheath insert.

23. The catheter-disinfecting system of claim 22, wherein one or more waveguide termini are incorporated into a sheath-insert body for circumferentially irradiating the lumen of the sheath insert, the lumen of the sheath insert coaxial with the lumen of the introducer sheath when the sheath insert is inserted into the introducer sheath.

24. The catheter-disinfecting system of claim 23, wherein the one-or-more waveguide termini are incorporated into the diffuser disposed in the sheath-insert body for circumferentially irradiating the lumen of the sheath insert from the diffuser of the sheath insert.

25. The catheter-disinfecting system of claim 23, wherein the one-or-more waveguide termini are incorporated into the sheath-insert body behind the diffuser for circumferentially irradiating the lumen of the sheath insert through the diffuser of the sheath insert.

26. The catheter-disinfecting system of claim 24, wherein the diffuser includes a plurality of dimples configured to scatter the germicidal radiation transmitted into the diffuser out into the lumen of the sheath insert.

27. The catheter-disinfecting system of claim 23, wherein the waveguide includes one or more optical fibers, the one-or-more waveguide termini being one or more termini of the one-or-more optical fibers, respectively.

28. The catheter-disinfecting system of claim 1, wherein the germicidal radiation is selected from broad spectrum ultraviolet (“UV”)-visible (“vis”) light, broad spectrum UV light, UVA light, UVB light, UVC light, blue light, and modulated light thereof, the modulated light being modulated with respect to frequency, power, duration, or a combination thereof.

29. The catheter-disinfecting system of claim 1, wherein the introducer sheath is splittable for removing the introducer sheath from the insertion site without having to remove the catheter from the insertion site.

30. A catheter-disinfecting system, comprising:
a catheter including a catheter tube, the catheter tube, an abluminal coating on the catheter tube, or both the catheter tube and the abluminal coating including a photosensitizer configured to produce disinfecting reactive oxygen species (“ROS”) when irradiated with excitation radiation;
an introducer sheath including:
a sheath body configured to be percutaneously inserted into an insertion site of a patient; and
a sheath hub coupled to a proximal portion of the sheath body; and
an excitation irradiation means for irradiating the catheter tube of the catheter with the excitation radiation as the catheter tube is inserted into a lumen of the introducer sheath, thereby producing the disinfecting ROS and disinfecting the catheter tube immediately before insertion into the insertion site.

31. The catheter-disinfecting system of claim 30, wherein the photosensitizer is incorporated into the catheter tube.

32. The catheter-disinfecting system of claim 30, wherein the photosensitizer is incorporated into the abluminal coating on the catheter tube.

33. The catheter-disinfecting system of claim 30, wherein the photosensitizer is selected from toluidine blue, methylene blue, rose bengal, and a porphyrin-based ROS-generating compound.

34. The catheter-disinfecting system of claim 30, wherein the excitation irradiation means includes one or more internal radiation sources incorporated into the introducer sheath, the one-or-more internal radiation sources configured to both produce and emit the excitation radiation into the lumen of the introducer sheath.

35. The catheter-disinfecting system of claim 30, further comprising a sheath-hub attachment configured to removably couple to the sheath hub, the excitation irradiation means including one or more internal radiation sources incorporated into the sheath-hub attachment, and the one-or-more internal radiation sources configured to both produce and emit the excitation radiation into a lumen of the sheath-hub attachment.

36. The catheter-disinfecting system of claim 30, further comprising a sheath-hub attachment configured to removably couple to the sheath hub, the excitation irradiation means including one or more external radiation sources operably coupled by a waveguide to the sheath-hub attachment, the one-or-more external radiation sources configured to produce the excitation radiation, the waveguide configured to transmit the excitation radiation, and either the waveguide or a diffuser operably coupled to the waveguide configured to emit the excitation radiation into a lumen of the sheath-hub attachment.

37. The catheter-disinfecting system of claim 30, further comprising a sheath insert configured to removably insert into the introducer sheath, the excitation irradiation means including one or more external radiation sources operably coupled by a waveguide to the sheath insert, the one-or-more external radiation sources configured to produce the excitation radiation, the waveguide configured to transmit the excitation radiation, and either the waveguide or a diffuser operably coupled to the waveguide configured to emit the excitation radiation into a lumen of the sheath insert.

38. A method of disinfecting a catheter, comprising:
inserting a catheter tube of the catheter into an introducer sheath percutaneously inserted into an insertion site of a patient; and
irradiating the catheter tube with radiation while inserting the catheter tube into a sheath body of the introducer sheath by way of a sheath hub, the irradiating of the catheter tube disinfecting the catheter tube immediately before insertion into the insertion site.

39. The method of claim 38, wherein the irradiating of the catheter tube with radiation includes irradiating the catheter tube with germicidal radiation, the germicidal radiation selected from broad spectrum ultraviolet (“UV”)-visible (“vis”) light, broad spectrum UV light, UVA light, UVB light, UVC light, blue light, and modulated light thereof, and the modulated light being modulated with respect to frequency, power, duration, or a combination thereof.

40. The method of claim 38, wherein the irradiating of the catheter tube with radiation includes irradiating the catheter tube with the excitation radiation, the catheter tube or an abluminal coating on the catheter including a photosensitizer configured to produce disinfecting reactive oxygen species (“ROS”) when irradiated with the excitation radiation.

41. The method of claim 38, wherein the irradiating of the catheter tube with radiation includes irradiating the catheter tube with one or more internal radiation sources incorporated into the introducer sheath, the one-or-more internal radiation sources configured to both produce and emit the radiation into the lumen of the introducer sheath.

42. The method of claim 38, wherein the irradiating of the catheter tube with radiation includes irradiating the catheter tube with one or more internal radiation sources incorporated into a sheath-hub attachment configured to removably couple to the sheath hub.

43. The method of claim 40, wherein the irradiating of the catheter tube with radiation includes irradiating the catheter tube with one or more external radiation sources operably coupled by a waveguide to a sheath-hub attachment removably coupled to the sheath hub, the one-or-more external radiation sources configured to produce the radiation, the waveguide configured to transmit the radiation, and either the waveguide or a diffuser operably coupled to the waveguide configured to emit the radiation into a lumen of the sheath-hub attachment.

44. The method of claim 40, wherein the irradiating of the catheter tube with the radiation includes irradiating the catheter tube with one or more external radiation sources operably coupled by a waveguide to a sheath insert removably inserted into the introducer sheath, the one-or-more external radiation sources configured to produce the radiation, the waveguide configured to transmit the radiation, and either the waveguide or a diffuser operably coupled to the waveguide configured to emit the radiation into a lumen of the sheath insert.
